(19) United States
(12) Patent Application Publication
(10) Pub. No.: US 2026/0276576 A1
(43) Pub. Date: Sep. 17, 2026

(54) Systems and Methods for Sensing Melatonin and Other Hormones

(71) Applicant: Siddhartha LLC, Sacramento, CA (US)

(72) Inventors: Preetam Anbukarasu, Edmonton (CA); Sangita Sridar, Edmonton (CA); Jyothirmayee Aravind Vishnusekhar, Madison, WI (US); Michele Guindani, Irvine, CA (US); Rohan Priyanka Calnaido, Irvine, CA (US)

(73) Assignee: Siddhartha LLC, Sacramento, CA (US)

(21) Appl. No.: 19/385,091

(22) Filed: Nov. 10, 2025

Related U.S. Application Data
(60) Provisional application No. 63/718,494, filed on Nov. 8, 2024.

(51) Int. Cl. G01N 27/327

(52) U.S. Cl. CPC G01N 27/3271

(57) ABSTRACT

An analyte detection system includes an amperometric electrode system including a working electrode, which has a sensing layer and a transduction layer, the sensing layer being chemically linked to the transduction layer and anchored to an anchoring layer to preserve an orientation of the sensing layer, the sensing layer configured to detect an analyte within a fluid medium based on an interaction event between the analyte and the sensing layer when the analyte is oriented consistent with the orientation of the sensing layer. The analyte detection system includes an inlet reservoir from which the fluid medium travels towards the sensing layer; and an inlet channel through which the fluid medium travels from the inlet reservoir towards the sensing layer. The analyte is oriented consistent with the orientation of the sensing layer when a larger diameter end of the sensing layer is oriented towards the inlet channel or the inlet reservoir.

### CROSS REFERENCE TO RELATED APPLICATIONS

This application claims the benefit under 35 U.S.C. § 119(e) of the U.S. Provisional Application Ser. No. 63/718,494 filed Nov. 8, 2024, the content of which is hereby incorporated by reference in its entirety.

### TECHNICAL FIELD

The present disclosure relates generally to detection of certain analytes within a medium such as a fluidic medium. Some aspects of the disclosure relate to detection of melatonin.

### Description of Related Art

Current methods of detecting certain analytes within a fluidic medium may have shortcomings. For example, mass spectrometry and high performance liquid chromatography have limitations of collecting and appropriately storing and transporting fluidic samples, and are not equipped for a non-laboratory setting. Radioimmunoassay and enzyme-linked immunosorbent assay (ELISA) have similar limitations and may be unable to quantify daytime melatonin levels under 1 pg/mL unable to quantify daytime melatonin levels less than 1 pg/mL, and limitations collecting and appropriately storing and transporting sample. Such limitations may increase a burden on a subject, while increasing the likelihood of inaccurate results.

### BRIEFR SUMMARY OF THE DISCLOSURE

According to various aspects of the disclosed technology, an analyte detection system may comprise an amperometric electrode system comprising a working electrode, the working electrode may comprise: a sensing layer and a transduction layer, the sensing layer being chemically linked to the transduction layer to preserve an orientation of the sensing layer, the sensing layer configured to detect a concentration of an analyte within a fluid medium based on one or more interaction events between the analyte and the sensing layer when the analyte is oriented consistent with the orientation of the sensing layer. The analyte detection system may comprise an inlet reservoir from which the fluid medium travels towards the sensing layer; and an inlet channel through which the fluid medium travels from the inlet reservoir towards the sensing layer, wherein the analyte is oriented consistent with the orientation of the sensing layer when a larger diameter end of the sensing layer is oriented towards the inlet channel or the inlet reservoir.

As a specific illustrative example, an analyte detection system (e.g., in FIG. 5A) may comprise an amperometric electrode system 525 comprising a working electrode 520. The working electrode may comprise a sensing layer (e.g., 114, FIG. 1 or FIG. 2) and a transduction layer (e.g., 116, 215, FIG. 2). The sensing layer 114 may be chemically linked to the transduction layer 116 or 215 to preserve an orientation of the sensing layer. As shown in FIGS. 1 and 2, the sensing layer 114 has an orientation such that its larger diameter end is oriented upward, away from the transduction layer 116 or 215 and towards a direction from which the fluid medium travels towards the sensing layer 114. The smaller diameter end is oriented downward, towards the transduction layer 116 or 215. As shown in FIG. 5A, the direction from which the fluid medium travels towards the sensing layer is from the inlet channel (e.g., 532) and the inlet reservoir (e.g., 530). The sensing layer (e.g., 114) may be configured to detect a concentration of an analyte within a fluid medium based on one or more interaction events between the analyte and the sensing layer when the analyte is oriented consistent with the orientation of the sensing layer (e.g., when the sensing layer 114 is oriented towards the inlet channel 532 and the inlet reservoir 530).

In some aspects, the sensing layer is unable to detect the analyte when the analyte is oriented inconsistently with the orientation of the sensing layer.

In some aspects, the amperometric electrode system comprises an outlet channel through which the fluid medium travels from the sensing layer and an outlet reservoir to which the fluid medium travels from the sensing layer, wherein the analyte is oriented inconsistently with the orientation of the sensing layer when a larger diameter end of the sensing layer is oriented towards the outlet channel or the outlet reservoir.

In some aspects, the analyte is oriented inconsistently with the orientation of the sensing layer when a smaller diameter end of the sensing layer is oriented towards the inlet channel or the inlet reservoir.

In some aspects, the analyte comprises melatonin and the sensing layer comprises a thiolized or functionalized calixarenes.

In some aspects, the sensing layer is anchored to an anchoring layer, the anchoring layer comprising a polyacrylic acid brush or a polyacrylic acid film.

In some aspects, the transduction layer comprises a conducting layer and a surface plasmon enhancing layer.

In some aspects, the conducting layer comprises graphite, single or multi walled carbon nanotubes, graphene nanosheets, graphene nanoplatelets, reduced graphene oxide (RGO), buckminsterfullerenes, boron nitride nanotubes, Mxenes, one-dimensional (1D) nanowires, nanotubes or two-dimensional (2D) nanosheets, metal nanoparticles or metal oxide nanoparticles, a film of noble metals, or metal oxide nanotubes.

In some aspects, the surface plasmon enhancing layer comprises a nanostructured noble metal.

In some aspects, the fluid medium comprises a saliva, a plasma, or urine.

In some aspects, a method comprises: directing a fluid medium containing an analyte from an inlet reservoir through an inlet channel towards a sensing layer of an amperometric electrode system; detecting, by an amperometric electrode system, a concentration of the analyte within a fluid medium based on one or more interaction events between the analyte and a sensing layer of the amperometric electrode system when the analyte is oriented consistent with an orientation of the sensing layer, wherein the analyte is oriented consistent with the orientation of the sensing layer when a larger diameter end of the sensing layer is oriented towards the inlet channel or the inlet reservoir, and wherein the sensing layer is chemically linked to a transduction layer of the amperometric electrode system to preserve the orientation of the sensing layer.

In some aspects, the sensing layer is unable to detect the analyte when the analyte is oriented inconsistently with the orientation of the sensing layer.

In some aspects, the method further comprises directing the fluid medium from the sensing layer towards an outlet reservoir through an outlet channel, wherein the analyte is oriented inconsistently with the orientation of the sensing layer when a larger diameter end of the sensing layer is oriented towards the outlet channel or the outlet reservoir.

In some aspects, the analyte is oriented inconsistently with the orientation of the sensing layer when a smaller diameter end of the sensing layer is oriented towards the inlet channel or the inlet reservoir.

In some aspects, the analyte comprises melatonin and the sensing layer comprises a thiolized or functionalized calixarenes.

In some aspects, the method further comprises anchoring the sensor layer to an anchoring layer to preserve the orientation of the sensor layer, wherein the anchoring layer comprises a polyacrylic acid brush or a polyacrylic acid film.

In some aspects, the transduction layer comprises a conducting layer and a surface plasmon enhancing layer.

In some aspects, the conducting layer comprises graphite, single or multi walled carbon nanotubes, graphene nanosheets, graphene nanoplatelets, reduced graphene oxide (RGO), buckminsterfullerenes, boron-nitride nanotubes, Mxenes, one-dimensional (1D) nanowires, nanotubes or two-dimensional (2D) nanosheets, metal nanoparticles or metal oxide nanoparticles, a film of noble metals, or metal oxide nanotubes.

In some aspects, the surface plasmon enhancing layer comprises a nanostructured noble metal.

In some aspects, the fluid medium comprises a saliva, a plasma, or urine.

### BRIEF DESCRIPTION OF THE DRAWINGS

The present disclosure, in accordance with one or more various aspects, is described in detail with reference to the following figures. The figures are provided for purposes of illustration only and merely depict typical or example aspects.

FIG. 1 illustrates an analyte detection environment with which aspects of the systems and methods disclosed herein may be implemented.

FIG. 2 illustrates an analyte detection environment with which aspects of the systems and methods disclosed herein may be implemented.

FIG. 3 illustrates an analyte sensor anchoring method with which aspects of the systems and methods disclosed herein may be implemented.

FIG. 4A illustrates an analyte detection system with which aspects of the systems and methods disclosed herein may be implemented.

FIG. 4B illustrates an analyte detection environment with which aspects of the systems and methods disclosed herein may be implemented.

FIGS. 5A and 5B illustrate an analyte detection system with which aspects of the systems and methods disclosed herein may be implemented.

FIG. 5C illustrates an alternative analyte detection inlet system with which aspects of the systems and methods disclosed herein may be implemented.

FIG. 6 illustrates an analyte detection system with which aspects of the systems and methods disclosed herein may be implemented.

FIG. 7A illustrates an analyte detection system with which aspects of the systems and methods disclosed herein may be implemented.

FIG. 7B illustrates an analyte detection calibration curve with which aspects of the systems and methods disclosed herein may be implemented.

FIGS. 8A-8C illustrate different perspectives of an analyte detection system with which aspects of the systems and methods disclosed herein may be implemented.

FIG. 9 illustrates an analyte measurement system with which aspects of the systems and methods disclosed herein may be implemented.

FIG. 10 illustrates a method of measuring analyte concentrations with which aspects of the systems and methods disclosed herein may be implemented.

FIG. 11A illustrates a transduction layer surface with which aspects of the systems and methods disclosed herein may be implemented.

FIG. 11B illustrates a beta-cyclodextrin functionalized gold surface with which aspects of the systems and methods disclosed herein may be implemented.

FIGS. 12A-12E illustrate scanning electron microscopy (SEM) images of beta-cyclodextrin with which aspects of the systems and methods disclosed herein may be implemented.

FIG. 13 illustrates assemblies containing a sensing layer (e.g., beta-cyclodextrin) combined with one or more transduction layers with which aspects of the systems and methods disclosed herein may be implemented.

FIG. 14 illustrates a computer processor with which aspects of the systems and methods disclosed herein may be implemented.

The figures are not exhaustive and do not limit the present disclosure to the precise form disclosed. Any relevant principles from any Figs. may be implemented in conjunction with other Figs.

### DETAILED DESCRIPTION

A non-invasive analyte detection system may be implemented in any setting including a non-laboratory setting, while being sufficiently sensitive and specific for clinical purposes. The analyte detection system resolves existing problems of analyte detection systems, which include lack of sensitivity, and being limited to implementation in a laboratory setting. The analyte detection system may obviate the need for strict protocols of obtaining and preserving fluidic samples from subjects which prevents potential inaccuracies from sample breakdown over time. In some aspects, the analyte detection system includes a melatonin detector or sensor (hereinafter “detector”) configured to detect melatonin stochastically in multiple fluid mediums within a fluidic sample, such as saliva, plasma, or urine. In some aspects, the melatonin detector is configured to detect dim light melatonin onset. Sensitivity may be especially important in such applications. Although the description focuses on melatonin detection, it is understood that other analytes besides melatonin may be detected.

In some aspects, the analyte detection system may be non-invasive and may include an architecture having different layers. The different layers may include a sensing layer and a transduction layer. The sensing layer may contain one or more interacting or scaffolding compounds such as calixarenes (e.g., thiolized or functionalized calixarenes such as thiolized beta-cyclodextrin), or other organic compounds or pore forming agents, which are configured to interact with one or more analytes (e.g., target analytes of interest) such as melatonin. Calixarenes may include beta-cyclodextrin, protoporphyrin IX, or alpha-hemolysin or hemin. Although the description focuses on beta-cyclodextrin, it is understood that other interacting compounds may be implemented in place of beta-cyclodextrin. In some aspects, the one or more interacting compounds may be nanoscopic, and may have pore size, orientation, affinity, oxidation potential, or other attribute that is compatible with or sensitive to one or more analytes to be detected. In some aspects, the sensing layer additionally or alternatively includes one or more antibodies (e.g., monoclonal antibodies) which may provide synergistic benefits of more effective or stronger interactions between the sensing layer and the one or more analytes.

In some aspects, the transduction layer contains a conducting layer or a surface plasmon enhancing layer. The conducting layer may include any one or combination of graphite, carbon, single/multi walled carbon nanotubes, graphene nanosheets, graphene nanoplatelets, reduced graphene oxide (RGO), buckminsterfullerenes, boron-nitride nanotubes, Mxenes, other one-dimensional (1D) nanowires, nanotubes or two-dimensional (2D) nanosheets or their hybrids, metals/metal oxide nanoparticles, smooth or wrinkled thin film deposition of noble metals, or metal oxide nanotubes. In some aspects, the conducting layer may have thickness between 1 nanometer (nm) to 100 nm, 1 nm to 50 nm, 1 nm to 25 nm, 1 nm to 20 nm, or any subrange or value therebetween. In some aspects, the conducting layer may have thickness of approximately 4 nm. In some examples, the conducting layer may be doped, such as using nitrogen, sulfur or boron. In some aspects, the surface plasmon enhancing layer includes a nanostructured layer such as a nanostructured noble metal (e.g., gold) nanolayer. In some aspects, additionally or alternatively, the surface plasmon enhancing layer includes noble metals such as silver (Ag), platinum (Pt) or palladium (Pd). Metals such as Au, Ag, Pt and Pd provide high electrical conductivity, catalytic activity and stability. Au may easily bind to thiol molecules, facilitating effective immobilization of biomolecules. Various sizes and shapes of Au nanoparticles can be utilized based on their plasmonic effects. For instance, nano sphere, nano rod, nanoflower, nanocage, nanocubes or nanostars may be utilized. In some aspects, a surface plasmon enhancing layer positioned to directly contact a conducting layer may prevent agglomeration of surface plasmon enhancing layer nanoparticles (e.g., gold nanoparticles) which would otherwise degrade sensor capabilities, and decrease tendency of the conducting layer (e.g., graphene) to stack.

The transduction layer may contain a substrate layer. In other aspects, the transduction layer may be devoid of a substrate layer. In some aspects, the substrate layer may be disposed separately from the transduction layer. The substrate layer may contain a support such as silicon dioxide, a dielectric material such as Mylar or polycarbonate, a porous membrane nitrocellulose, or fiberglass. In some aspects, the different layers contain an anchoring layer or a stochastic layer, which may contain a film such as polyacrylic acid or polymer polyacrylic acid that is configured to anchor the sensing layer. In some aspects, the anchoring layer may be part of the sensing layer, or separate from the sensing layer.

As a specific exemplary implementation, the conducting layer may include graphene. Graphene sheets have benefits of desirable electrical, mechanical and structural characteristics due to their specific nanostructure configuration. The high surface area of graphene can provide numerous active sites for binding of analytes. Further, graphene could facilitate fast electron transfer between the analyte and an electrode acting as a conducting channel for stochastic measurements.

In some aspects, any of the different layers (e.g., the sensing layer and the transduction layer) form a working electrode, as part of a multi-electrode sensor which may also include a reference electrode and a counter electrode. In some aspects, the reference electrode includes an silver/silver chloride (Ag/AgCl) electrode or a silver (Ag) deposit, or any other suitable materials on a substrate. In some aspects, the reference electrode may or may not contain graphene. In some aspects, the counter electrode may contain a platinum (Pt) wire, Pt mesh, Pt sheet, Graphene or carbon electrode, or any other suitable materials.

In some aspects, a conducting shield such as aluminum, medical grade stainless steel or silicone or any other conductive material can be used as a Faraday's cage to encapsulate the analyte detection system, or as part of the analyte detection system, to eliminate or mitigate electromagnetic interference.

In some aspects, a few nanometer thick (e.g., 1-10 nm) graphene layer may be deposited on the working electrode, followed by deposition of a thin gold film by sputtering. Chains of polyacrylic acid (PAA) brushes in the form of hexamers or tetramers may cross link beta-cyclodextrin and be used to reduce the fouling of an electrode surface by contaminants, because the PAA brushes may be in motion. It may be more difficult for the contaminants to attach to the surface of the PAA brushes. In some aspects, the PAA brushes are replaced with long chain polymers. In some aspects, additionally or alternatively, a thin layer of graphene is deposited on the working electrode, with a few nanometer thick gold film deposited on top of the graphene. Thiol functionalization may be employed to anchor the beta-cyclodextrin on top of the gold film. PAA may reduce fouling and extend the lifetime of operation of the analyte detection system. In some aspects, gold nanoparticles instead of a gold film and the beta-cyclodextrin has been anchored on top of the gold nanoparticles as part of a stochastic layer.

In some aspects, an analyte detection system for melatonin includes a microneedle based sensor. The working electrode, as described above, can be fabricated using Au nanoparticle functionalized graphene nanomaterials and a pore forming layer for stochastic detection. Any other 1D, 2D nanomaterials and polymer composites can be utilized in this construction. Fabrication may be performed with or without nitrogen or other elemental doping before, with or after plasma treatment or other kinds of chemical functionalization to decrease the hydrophobicity of graphene or to create anchor points. Fabrication may include carbonyl group functionalization, for example with gold sputtering, chemical reduction or self assembly, or further functionalization in the instance of titanium oxide. Functionalization may include application of polyacrylic acid or polymer polyacrylic acid, or beta-cyclodextrin solution. Pendant moieties may be added to adjust charge or adjust size of the beta-cyclodextrin pores.

Several different approaches may be utilized to prepare the working electrode. In a first approach, a few layer graphene was deposited on top of the screen printed electrode by chemical vapor deposition (CVD) technique. The graphene coated electrode may be masked and sputter coated with Au on the working electrode sensor surface by RF sputtering. The third step was a thiol functionalization step to anchor the beta-cyclodextrin as the stochastic layer. In a second approach, graphite oxide (GO) was prepared according to a modified Hummers method. Graphene was prepared by exfoliation of graphite oxide by one of the exfoliation techniques such as thermal or chemical exfoliation. Required amount of PVC binder was dissolved in 0.5 mL DMF and a certain amount of graphene was dispersed in the solution by ultrasonication for a certain amount of time (e.g., approximately 30 minutes) to get a uniform suspension. 3 μl of the graphene ink was then drop casted on top of the solid composite polymer electrolyte (SCPE) and allowed to dry at room temperature (e.g., between 10 and 40 degrees Celsius, any subrange, or any temperature within the aforementioned range, or any other suitable range). This was then followed by sputter deposition of the Au film and thiolization process to anchor beta-cyclodextrin.

Another chemical approach may involve in situ-reduction of graphite oxide and gold salt. An amount of graphite oxide (GO) solution may be added into a volumetric flask followed by the addition of required amount of 1.0 mM chloroauric acid (HAuCl₄) solution and then diluted with 0.1 M (pH 9.2) phosphate buffer solution (PBS). The final solution had a GO concentration of 1.0 mg/mL. After that, the solution was sonicated for around 30 seconds. A bare screen printed electrode (SPE) may be washed with ethanol and deionized water. Then, 5 μL of the as-prepared solution may be dropcasted on the SPE and allowed to dry overnight at 25° C. The SPE may be electrodeposited using a Cyclic voltammogram at potentials from 0V to −1.5V in a 0.1 M potassium chloride (KCI) solution in order to simultaneously reduce the GO to rGO and the Au³⁺ ions to gold nanoparticle composite. By varying the concentration of GO, various GO/Au nanoparticle modified electrodes may be fabricated.

As another approach, Au-Nitrogen graphene (Au/N-G) based biosensors may be fabricated. In the first method, an ink of N-G in one of the solvents such as deionized (DI) water/DMF/IPA may be prepared by sonication. The resulting slurry may be spin coated or drop casted on top of the electrode. After drying, Au deposition may occur at a sputter chamber, followed by thiolization process to anchor beta-cyclodextrin. Additionally or alternatively, an amount of N-Graphene may be taken in a container, and 2:1 vol. ratio of ethylene glycol-water solution may be added. To this, an amount of HAuCl₄ 3H₂O aqueous solution (prepared by dissolving 0.1 g of gold salt in 10 ml DI water) was added. The container may be placed on a hot plate with magnetic stirring and reduction reactions were performed under reflux conditions (at approximately 125° C.) for a given amount of time such as around two hours. The solution was then centrifuged and washed with de-ionized water a certain number of times (e.g., 4-5 times) and finally dried at approximately 60° C. This Au nanoparticles dispersed N-Graphene may be been used to coat the SPE surface before the thiolization process. In some aspects, an in situ reduction plasma method involves the reduction of GO-gold salt deposit atop the SPE while exposed to plasma under the following conditions: gas pressure of 0.6 Pa, RF power of 10 W and flowrate of 2 standard cubic centimeters per minute of nitrogen gas. This facilitated the simultaneous reduction of GO to graphene, gold salt to gold as well as N-doping in a single process.

High resolution scanning electron microscopy, transmission electron microscopy, X-ray photo electron spectra, and atomic force microscopy may confirm the formation of different layers on the working electrode, thickness of graphene nanosheets as well as the thickness of metal film deposition.

In some aspects, a potentiostat is used for the electrochemical studies and testing of the fabricated sensors. Quantitative and real-time monitoring of the biosensor electrodes may be performed by chronoamperometry. The prepared electrodes may have been conditioned by washing in common solvents and then scanning in a specific potential window by Cyclic voltammetry prior to amperometric measurements. Baseline measurements may be carried out initially with phosphate buffer with no analyte added. With different concentrations of melatonin, chronoamperometric curves have been recorded and one or more calibration relationships have been employed such as: 1 /tₒₙ=a+b×Conc. ₘₑₗₐₜₒₙᵢₙ. The amount of melatonin may be obtained by inserting the ton values in the above calibration equation. tₒₙ may refer to a pulsed on-time (e.g., corresponding to a time period in which the melatonin is binding to a wall of a channel of the beta-cyclodextrin), and a and b being constants. Additionally or alternatively, impedance measurements may be carried out to study the electrical properties and can be used to calibrate.

The analyte detection system may be encapsulated by a microfluidic system. The microfluidic system may contain Tesla valves, diffuser-nozzle elements, or capillary action valves to enhance the flow of melatonin containing fluid to the sensing region of the working electrode containing the calixarenes.

In some aspects, the analyte detection system enables real-time, at-home assessment of circadian rhythms using a fundamentally new, non-invasive approach using a novel graphene-based microfluidic thin film sensor which enables high-resolution, stochastic electrochemical detection of salivary melatonin in real time eliminating the need for manual sampling. Current salivary melatonin detection methods such as liquid chromatography-mass spectroscopy, RIA, ELISA and HPLC may not permit multiple saliva sampling and may require sample pre-treatment, among other limitations. Thus, the analyte detection system overcomes existing problems with current salivary melatonin detection methods. Electrochemical biosensors on the other hand offer high sensitivity, selectivity and at home detection.

Use of high surface area graphene could provide large number of binding sites for the target biomolecule, thereby improving the sensitivity of the biosensor. Also, the high electrical conductivity of graphene facilitates fast electron transfer between the analyte and the sensor electrode, leading to quick response times and high resolution detection. With high surface area and fast electron transfer kinetics, ultralow detection of melatonin is achieved. Moreover, the inherent flexibility and mechanical robustness of graphene contributes to the fabrication of the current analyte detection system, which may be implemented as a flexible and wearable biosensor for continuous, non-invasive detection of salivary melatonin.

The analyte detection system may be implemented in a non-laboratory setting, such as an at home point of care salivary melatonin testing device capable of testing serial saliva samples. As previously alluded to, the analyte detection system could be fabricated using any kind of 1D nanomaterials such as nanotubes, nanowires, nanofibers, and nanorods or 2D nanosheets, or nanoplatelets. Modification of this nanomaterial may be carried out using metal nanoparticles such as Au, Ag, Pt, Pt-Au, Pd etc to improve the surface area and electron transfer kinetics. Conductive polymers also can be used along with these nanomaterials for modifying the electrode to achieve desired performance. Both sputtering and chemical reduction of salt to metal to disperse metal/metal oxide nanoparticles on 1D or 2D nanomaterials can be employed. Thin thiolated poly ethylene glycol (PEG), PAA or other zwitterionic polymers can be self assembled on the gold layer to reduce/eliminate the fouling of the electrode. Stochastic layers can be fabricated using protoporphyrin IX, or alpha-hemolysin or hemin. In another embodiment, biorecognition elements such as antibodies and aptamers can be functionalized with nanostructures for high sensitive and selective detection of melatonin in saliva and to measure DLMO.

In some aspects, the analyte detection system may be implemented as an at home point of care urinary 6-sulfatoxymelatonin (aMT6s) testing device capable of testing serial urine samples. The synergistic effect of various nanoparticles (Ag, Au, Pt, Pd, Pt-Au etc) and 1D/2D nano materials can be explored for the fabrication of a sensor for the detection of melatonin in urine samples. To improve selectivity in complex biological matrices and to prevent interference from non-target molecules the electrode can be functionalized with monoclonal antibodies as a selective molecular recognition element for melatonin. The fabricated sensor can be used for sampling urine samples at given intervals such as every two to eight hours for a given window of time such as 24 to 48 hours or the first morning urine sample. The obtained aMT6s levels can then be corrected for creatinine to account for intraindividual variability.

In some aspects, the analyte detection system may be implemented as a microneedle filament that can be functionalized for sensing melatonin in different mediums such as plasma or interstitial fluid. A filament or manifold such as a nanostructured filament capable of selective detection of melatonin using highly conductive nanomaterials and melatonin recognition layer can be fabricated as the working electrode. Polymer based antifouling coating could ensure long term performance and built in microfluidic channel ensures continuous and improved flow of plasma to the sensing area of the working electrode. Ag/AgCl can be used as the reference electrode and platinum as counter electrode. The sensor could be packaged using flexible polymer substrate.

In some aspects, the analyte detection system may be implemented as part of a kit, which may include instructions regarding care and use as well as recommended protocol regarding factors effecting sampling and melatonin such as lighting, periods to avoid eating or drinking, position change during automated sampling preceding bedtime. The analyte detection system may be part of processing circuitry that collects data and transmits data to a device (e.g., a smartphone) where data can be viewed at latest the following day utilizing an application as well as feedback regarding movement and light exposures with integration of data from accelerometers or actigraphy, light sensors or photodetectors, or thermometers.

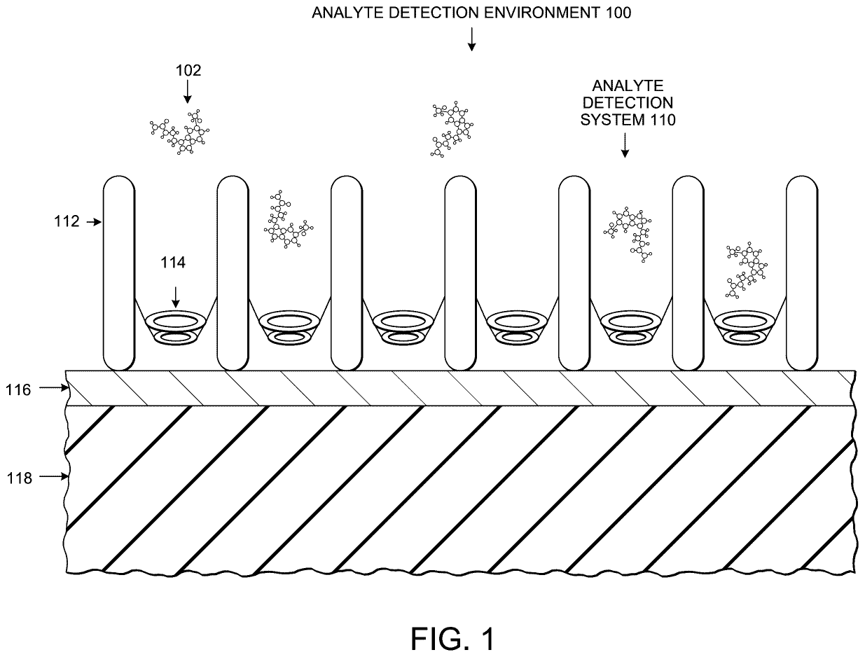

FIG. 1 illustrates an analyte detection environment 100 that includes one or more analytes 102 such as melatonin and an analyte detection system 110 configured to detect a melatonin concentration within a fluidic sample, such as saliva, plasma, or urine. It is understood that any reference to an analyte detection system may refer to a portion or an entirety of an analyte detection system. For example, the analyte detection system 110 illustrated in FIG. 1 may be implemented as components of another analyte detection system which may include additional components. The analyte detection system 110 may include any of a sensing layer 114 including one or more calixarenes (e.g., beta-cyclodextrin), an anchoring layer 112 that is configured to anchor the sensing layer 114, a transduction layer 116, and a substrate layer 118. In some aspects, the anchoring layer 112 includes a film such as polyacrylic acid or polymer polyacrylic acid. In some aspects, the transduction layer 116 includes a conducting layer. The conducting layer may include any one or combination of graphite, carbon, single/multi walled carbon nanotubes, graphene nanosheets, graphene nanoplatelets, reduced graphene oxide (RGO), buckminsterfullerenes, boron-nitride nanotubes, Mxenes, other one-dimensional (1D) nanowires, nanotubes or two-dimensional (2D) nanosheets or their hybrids, metals/metal oxide nanoparticles, smooth or wrinkled thin film deposition of noble metals, or metal oxide nanotubes. In some examples, the conducting layer may be doped, such as using nitrogen, sulfur or boron. The substrate layer 118 may contain a support such as silicon dioxide, a dielectric material such as Mylar or polycarbonate, a porous membrane nitrocellulose, or fiberglass. In FIG. 1, only one analyte 102 is labelled for simplicity although there are multiple analytes shown. Also, only one component or portion of the anchoring layer 112 and the sensing layer 114 is labelled for simplicity.

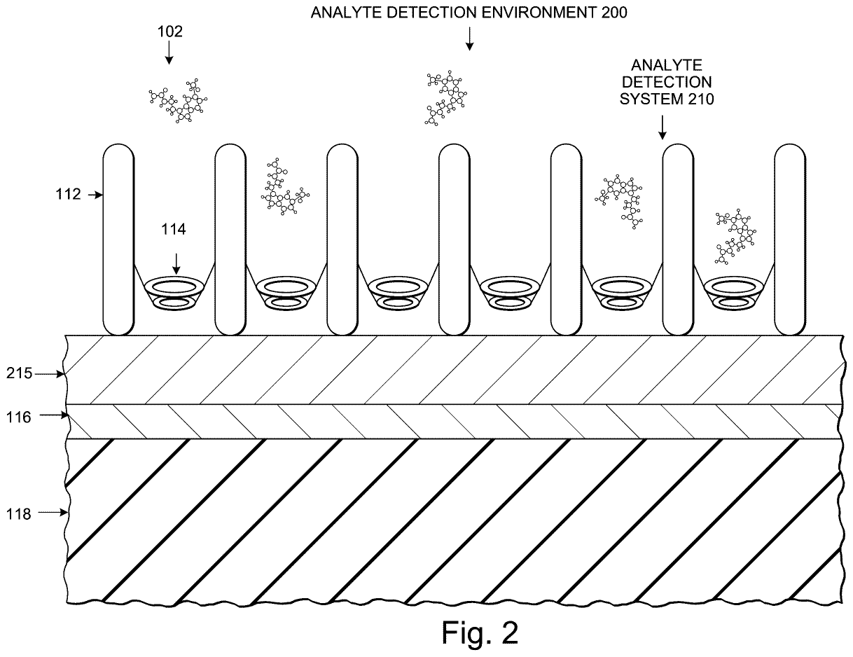

FIG. 2 illustrates an analyte detection environment 200. Any relevant aspects of analyte detection environment 100 may be applicable to analyte detection environment 200. The analyte detection environment 200 includes the one or more analytes 102 such as melatonin and an analyte detection system 210 configured to detect a melatonin concentration within a fluidic sample, such as saliva, plasma, or urine. The analyte detection system 210 may include any of the sensing layer 114, the anchoring layer 112, an additional transduction layer 215 which may include increase the sensitivity of detection, the transduction layer 116 and the substrate layer 118. In some aspects, the additional transduction layer 215 includes a surface plasmon enhancing layer. In some aspects, the sensing layer 114 may be linked, via a chemical linker, to the surface plasmon enhancing layer. In some aspects, the sensing layer 114 may cover the surface plasmon enhancing layer or the transduction layer 116 at a high surface density. For example, the sensing layer 114 may cover the surface plasmon enhancing layer or the transduction layer 116 with a surface coverage or surface density of between approximately 25 percent to 100 percent, 35 percent to 100 percent, 45 percent to 100 percent, 50 percent to 100 percent, 50 percent to 90 percent, 50 percent to 80 percent, 55 percent to 70 percent, 45 percent to 80 percent or 90 percent, 35 percent to 80 percent or 90 percent, or 25 percent to 80 percent or 90 percent, or any subrange or value therebetween. In some aspects, the sensing layer 114 may have a specific orientation. For example, if the sensing layer 114 includes beta-cyclodextrin, a larger diameter portion of the beta-cyclodextrin may be facing a direction from which a fluid is flowing. The larger diameter portion of the beta-cyclodextrin may have a pore size of approximately 1.4 nanometers (nm). Proper orientation may be needed in order for the beta-cyclodextrin to interact with melatonin. Otherwise, if the beta-cyclodextrin has an incorrect or inconsistent orientation (e.g., flipped over 180 degrees), the melatonin may be unable to interact with beta-cyclodextrin and therefore unable to be detected. In some aspects, the beta-cyclodextrin may be anchored to the anchoring layer 112 at a specific orientation or position to prevent the orientation from flipping over 180 degrees. In some aspects, the anchoring layer 112 has proper dimensions in order to minimize steric hindrance of other analytes that would otherwise interfere with interaction events between the beta-cyclodextrin and the melatonin. This may mean that a smaller diameter portion is prevented from facing a direction from which a fluid is flowing. In some aspects, the beta-cyclodextrin includes one or more other linkages to the surface plasmon enhancing layers. The linkages may be oriented in order to prevent an improper orientation of the beta-cyclodextrin. The preserving of proper orientation of the beta-cyclodextrin via one or more linkages, such as to the surface plasmon enhancing layers and the anchoring layer 112, constitutes a technical benefit resulting in increased binding affinity and accuracy of measurement of analytes such as melatonin. Meanwhile, the linkages between the beta-cyclodextrin and the surface plasmon enhancing layers or between the beta-cyclodextrin and the anchoring layer 112 may have a limited degree of freedom of movement, which further enhances binding affinity between melatonin and the beta-cyclodextrin while preventing the beta-cyclodextrin from having improper orientation. Such anchoring and preserving of proper orientation also enhances shelf life of the analyte detection system 200.

In some aspects, the surface plasmon enhancing layer includes a nanostructured layer such as a nanostructured gold nanolayer. In some aspects, additionally or alternatively, the surface plasmon enhancing layer includes noble metals such as gold (Au), silver (Ag), platinum (Pt) or palladium (Pd). Metals such as Au, Ag, Pt and Pd provide high electrical conductivity, catalytic activity and stability. Au may easily bind to thiol molecules, facilitating effective immobilization of biomolecules. Various sizes and shapes of Au nanoparticles can be utilized based on their plasmonic effects. For instance, nano sphere, nano rod, nanoflower, nanocage, nanocubes or nanostars may be utilized. The analyte detection system 110 may be configured to detect different interaction events between the analyte 112 (e.g., melatonin) and the sensing layer 114. In some aspects, the sensing layer 114 has a pore size, affinity, or orientation that matches corresponding attributes of the analyte 112. In some aspects, the sensing layer 114 may detect a plurality of interaction events in a cascading fashion due to the short time interval between different interaction events of different analyte particles to the sensing layer. In some aspects, the sensing layer 114 facilitates effective detection of melatonin at high sensitivity, with low limit of detection (LOD) and low limit of quantification (LOQ), while limiting baseline drift and increasing dynamic range. Meanwhile, the transduction layer 116 or the additional transduction layer 215 facilitates a detectable signal amplitude while reducing signal to noise ratio (SNR), reducing baseline drift, and reducing a noise floor.

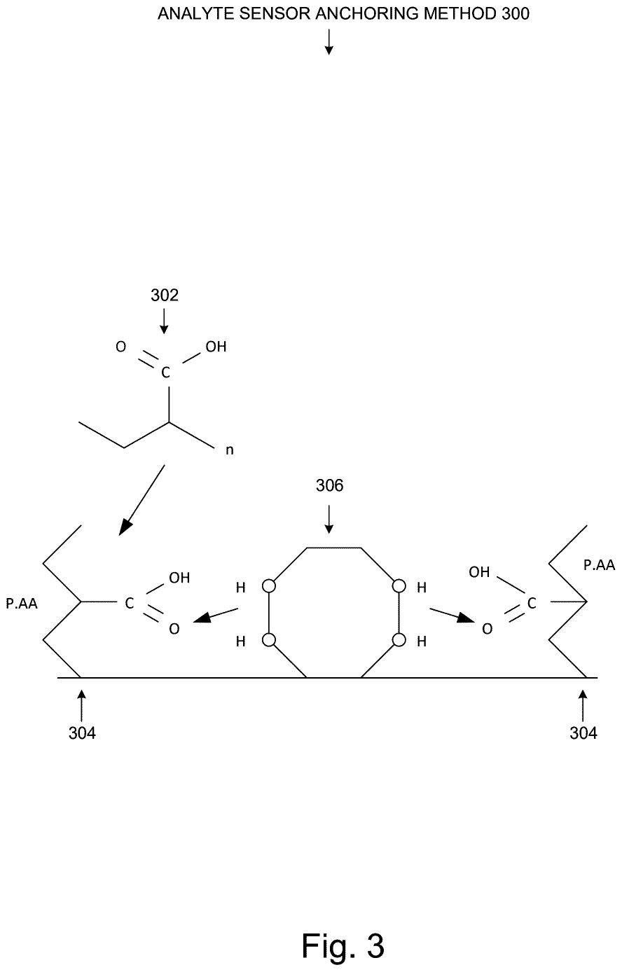

FIG. 3 illustrates an analyte sensor anchoring method 300. Any relevant aspects of analyte detection environment 100 or 200 may be applicable to analyte sensor anchoring method 300. In FIG. 3, an analyte sensor anchoring agent or compound 304, such as polyacrylic acid (PAA), may be formed, for example, by polymerization of monomeric precursors 302. The analyte sensor anchoring agent or compound 304 may attach strongly to analyte sensor compounds 306, which may be implemented as the sensing layer 114 or as part of the sensing layer 114. In some aspects, the analyte sensor compounds 306 attach to the analyte sensor anchoring compound 304 via hydrogen bonding or electron sharing or electron exchange. In this manner, the analyte sensor anchoring method 300 may securely anchor the analyte sensor anchoring compound 304 so that the analyte sensor anchoring compound 304 is not floating freely. Additionally, the analyte sensor anchoring method 300 fosters a hydrophilic zone due to hydrogen bonding of the analyte sensor anchoring compound 304 and the analyte sensor compounds 306. This increases the accuracy of detection of an analyte and binding affinity of the melatonin to the analyte sensor compounds 306. Furthermore, due to the negatively charged surface of the analyte sensor anchoring compound 304, fouling is prevented. Additionally, the mobile nature of the analyte sensor anchoring compound 304, which may be manifested as pH-responsive grafted layers, may further enhance anti-fouling properties of the analyte sensor anchoring compound 304.

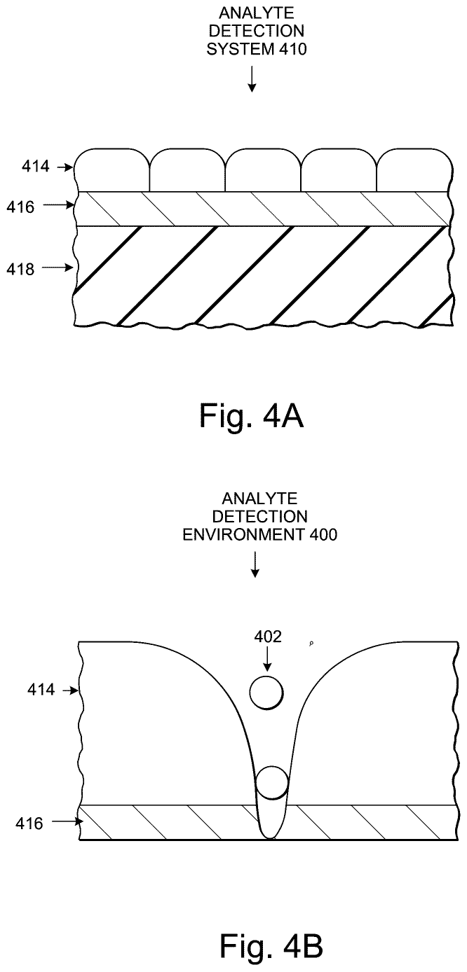

FIG. 4A illustrates an analyte detection system 410 which includes a sensing layer 414, a transduction layer 416, and a substrate layer 418. In some aspects, the sensing layer 414, the transduction layer 416, and the substrate layer 418 may be implemented as the sensing layer 114, the transduction layer 116, and the substrate layer 118, respectively, as described in FIGS. 1 and 2. FIG. 4B illustrates an analyte detection environment 400 which additionally includes one or more analytes 402. FIG. 4B illustrates that the analytes 402 interact with one or more pores, crevices, or other features within the sensing layer 414. Upon each analyte 402 interacting with (e.g., contacting, binding, or fitting within one or more features (e.g., pores) of) the sensing layer 414, the analyte detection system 410 detects an interaction event.

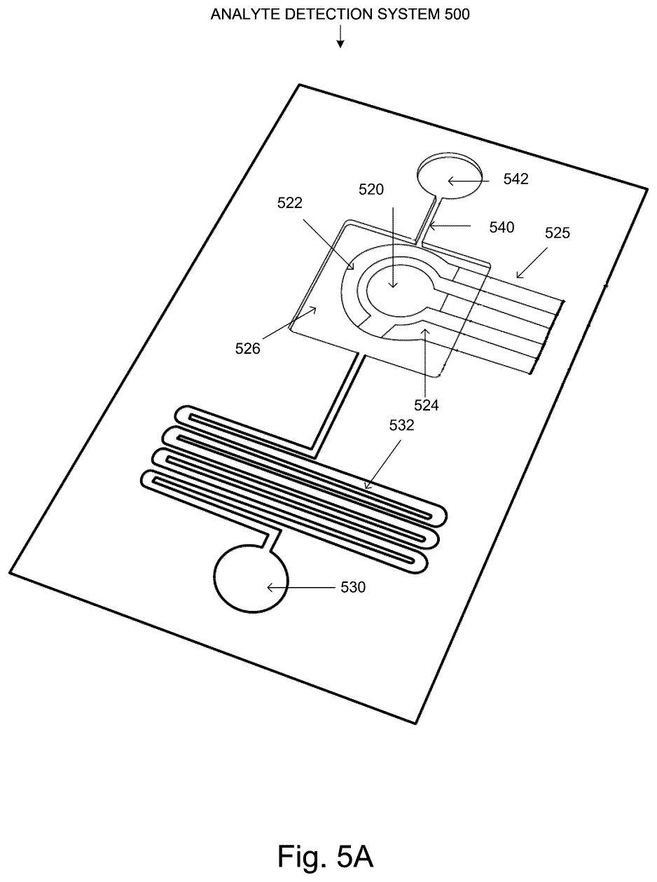

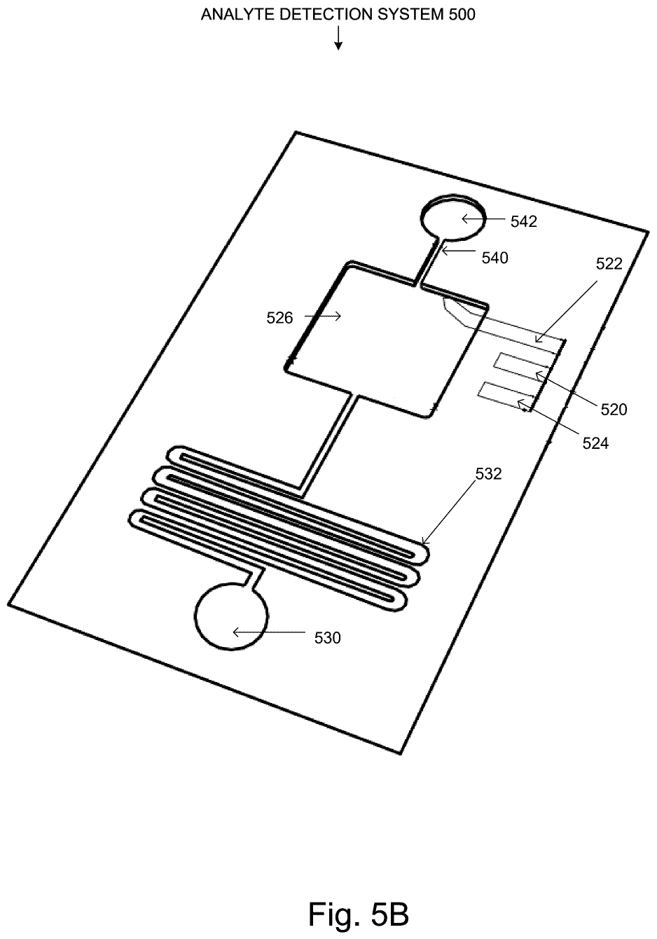

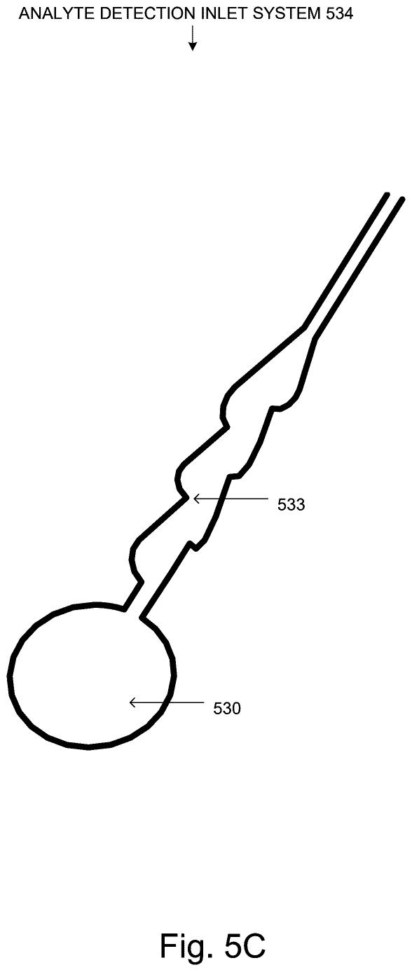

FIGS. 5A and 5B illustrate an analyte detection system 500 which includes an inlet reservoir 530, one or more inlet channels 532, an electrode system 525 (e.g., an amperometric or chronoamperometric electrode system) containing a working electrode 520, a counter electrode 522, and a reference electrode 524, a cavity 526 which may partially or entirely contain the sensor components of the electrode system 525, one or more outlet channels 540, and an outlet reservoir 542. In some aspects, the electrode system 525 may include a top laminate or a bottom laminate layer. FIG. 5A illustrates a top view of the electrode system 525 while FIG. 5B illustrates a bottom view. Relevant aspects of previously described Figs. such as FIGS. 1, 2, 3, or 4 may be implemented in conjunction with FIGS. 5A and 5B. In some aspects, the working electrode 520 contains one or more aforementioned layers of the analyte detection system 500, such as the sensing layer 114, the transduction layer 116, or the additional transduction layer 215. In some aspects, the working electrode 520 contains the anchoring layer 112. Thus, the sensing layer 114, the transduction layer 116, or the additional transduction layer 215 may constitute the working electrode 520. The working electrode 520 may be disposed separately from the counter electrode 522 and the reference electrode 524. The counter electrode 522 and the reference electrode 524 may close a circuit formed by the working electrode 520.

In some aspects, fluid which contains analytes (e.g., the analytes 112) may be received in the inlet reservoir 530 and may be directed to commence flowing substantially unidirectionally via the inlet channels 532, for example, by capillary forces or by pumping forces. Once the fluid enters the cavity 526, the fluid may contact the electrode system 525, in particular, the working electrode 520, which may sense analytes within the fluid. In some aspects, at least a portion of the electrode system 525, in particular, the working electrode 520, may contact a surface of the cavity 526. In other aspects, at least a portion of the electrode system 525, in particular, the working electrode 520, may not contact a surface of the cavity 526 and be offset (e.g., in a depth direction) from a surface of the cavity 526. The fluid may proceed to flow towards the outlet channels 540 and towards the outlet reservoir 542. As shown in FIG. 5A, the direction from which the fluid medium travels towards the sensing layer is that of the inlet channel (e.g., 532) and the inlet reservoir (e.g., 530). The sensing layer 114 (e.g., part of the working electrode 520) may be configured to detect a concentration of an analyte within a fluid medium based on one or more interaction events between the analyte and the sensing layer when the analyte is oriented consistent with the orientation of the sensing layer (e.g., when the sensing layer 114 is oriented towards the inlet channel 532 and the inlet reservoir 530.)

FIG. 5C illustrates an alternative analyte detection inlet system 534, which is distinct from the inlet reservoir 530 and the inlet channels 532 illustrated in FIGS. 5A and 5B. Instead of the inlet channels 532, which wind in a serpentine manner, other shapes or dimensions of the inlet channels may be implemented, such as inlet channels 533.

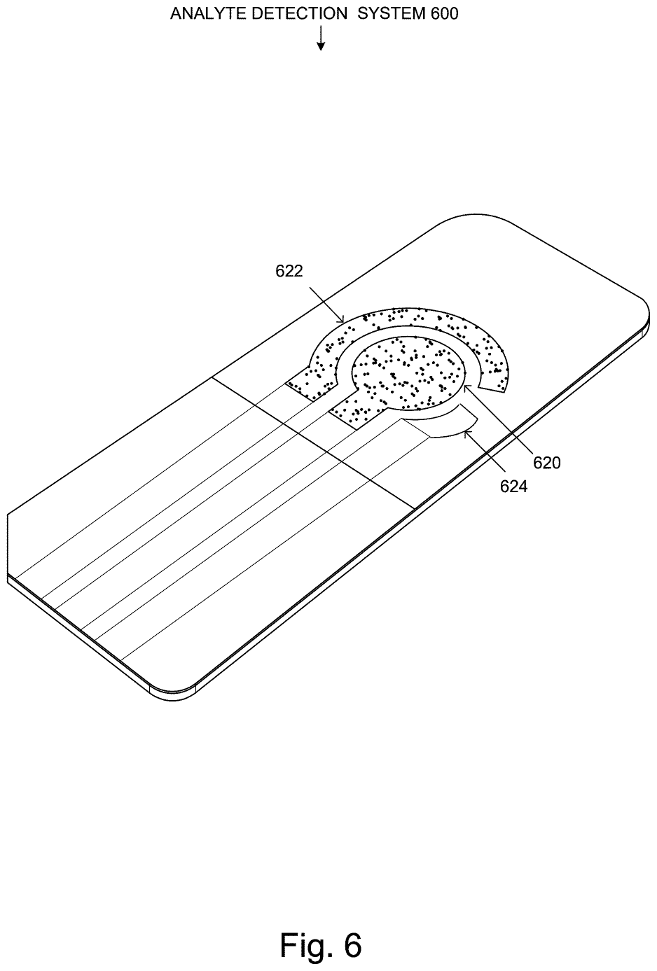

FIG. 6 illustrates an analyte detection system 600 which includes an electrode system that may be implemented in a same or similar manner as the electrode system 525. In some aspects, the electrode system illustrated in FIG. 6 includes a working electrode 620, a counter electrode 622, and a reference electrode 624. In some aspects, at least a portion of the electrode system 525, in particular, the working electrode 620, may be imprinted or otherwise positioned atop a substrate layer (e.g., the substrate layer 118).

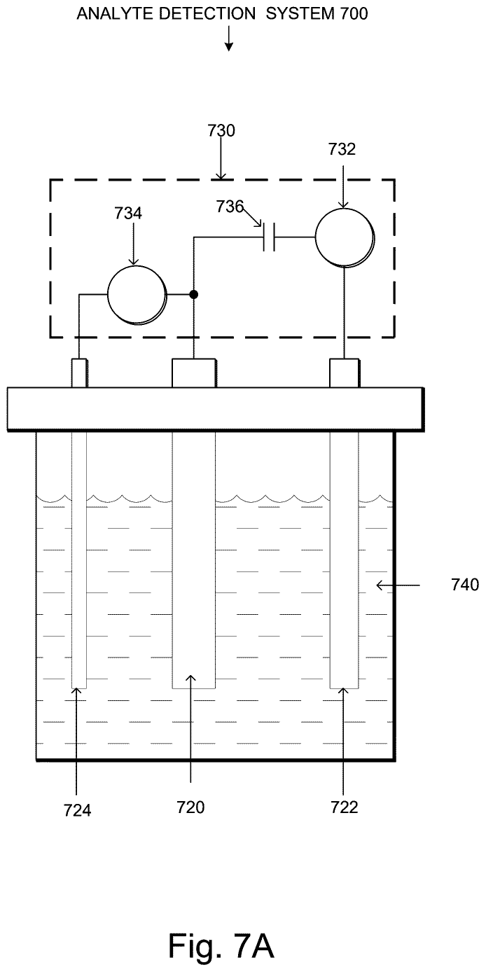

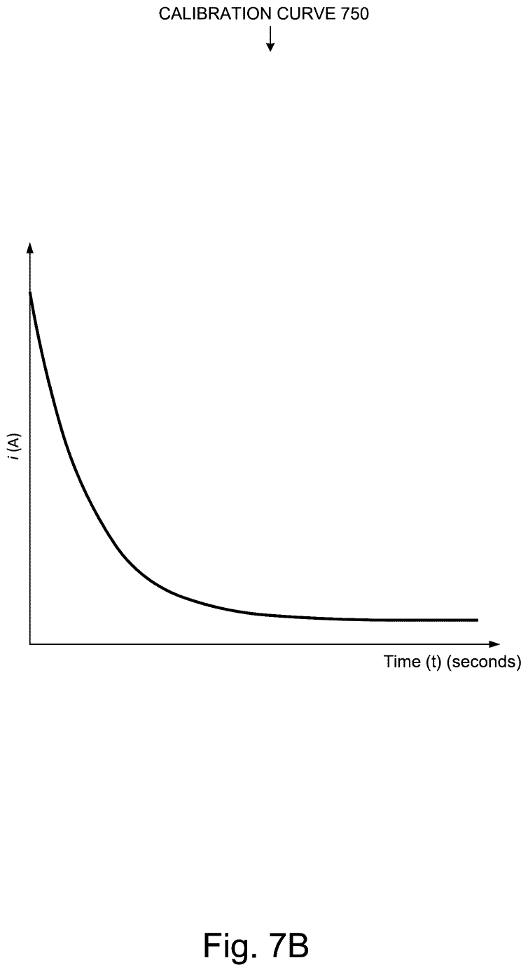

FIG. 7A illustrates an analyte detection system 700 which includes an electrode system that may be implemented in a same or similar manner as the previously described electrode system 525. In some aspects, the electrode system illustrated in FIG. 7A includes a working electrode 720, a counter electrode 722, and a reference electrode 724 that forms a closed circuit. The analyte detection system 700 may include a potentiostat 730, which measures electrical parameters such as current or voltage within the closed circuit. For example, the potentiostat 730 may set a given voltage 736 which may constitute a driving voltage. An ammeter 734 may measure one or more currents. Alternatively, a voltmeter 732 may measure one or more voltages. In some examples, the potentiostat 730 may, upon an interaction event of melatonin which is sensed by the working electrode 720, a difference in current of the working electrode 720 and current of the counter electrode 722 arises. This may cause a current flowing between the working electrode 720 and the counter electrode 722, which may cause a working electrode voltage difference between the working electrode 720 and the reference electrode 724 to deviate from a counter electrode voltage difference between the counter electrode 722 and the reference electrode 724. Without an interaction event, the working electrode voltage difference and the counter electrode voltage difference may be approximately the same. An amount of change in the current may be correlated to a number of interaction events or a concentration of melatonin.

FIG. 7B illustrates an analyte detection calibration curve 750. The calibration curve 750 illustrates a electrical current measured over a period of time, under an assumption that no interaction events (e.g., melatonin interacting with the working electrode 720) occur. The electrical current may decrease over time due to electrostatic interactions or layer or bilayer degradation over time, such as degradation of the sensing layer 114, the transduction layer 116, or the additional transduction layer 215. In some aspects, any detected currents (e.g., current spikes) resulting from interaction events (e.g., burst events) may be normalized with respect to the calibration curve 750. In some aspects, a high frequency (e.g., on an order of kiloHertz (kH)) may resolve single spike events.

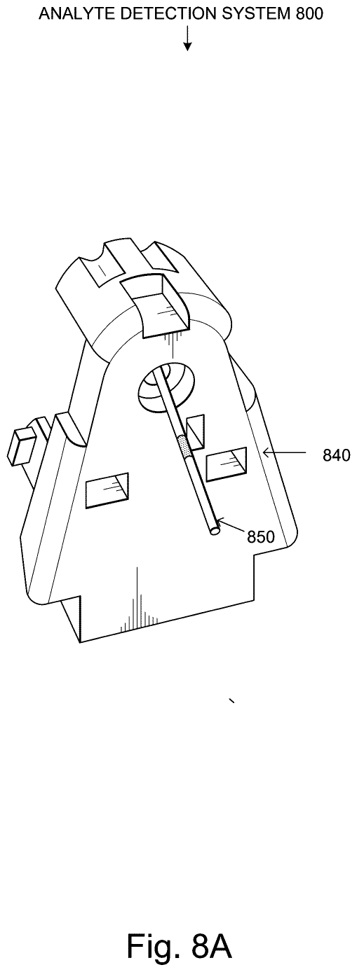

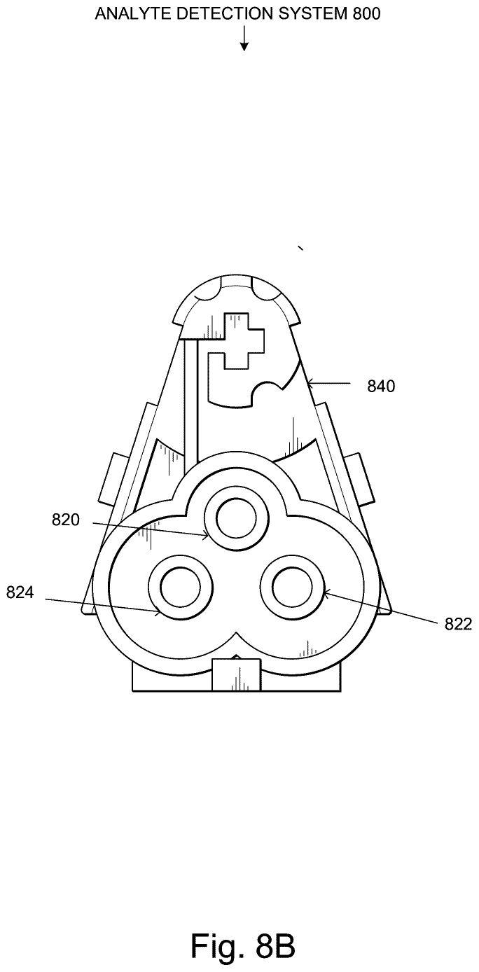

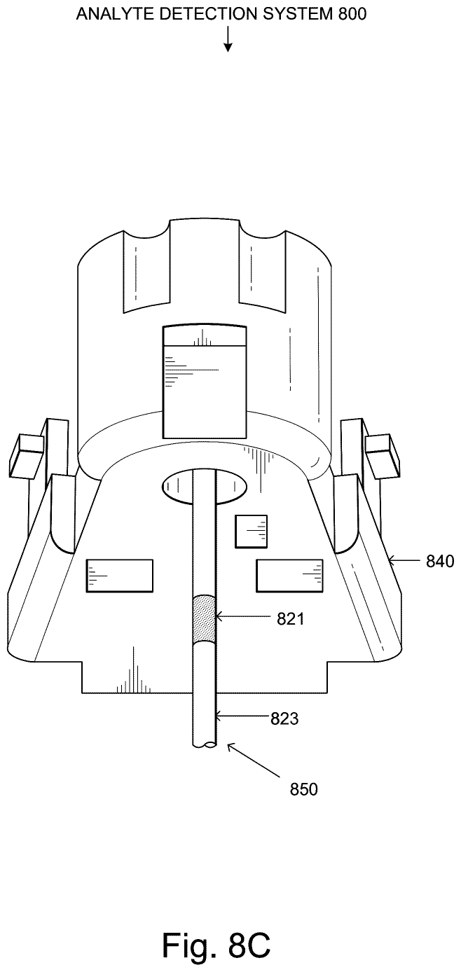

FIGS. 8A-8C illustrate different perspectives of an analyte detection system 800. In some aspects, the analyte detection system 800 includes a filament 850 (e.g., a functionalized filament) that is housed or enclosed by a housing, enclosure, or manifold (hereinafter “housing”) 840. In analyte detection system 800 may be configured to detect one or more analytes such as melatonin within a plasma fluid. In some aspects, the filament 850 includes electrode contacts such as a working electrode contact 820, a counter electrode contact 822, and a reference electrode contact 824, as illustrated in FIG. 8B. In some aspects, as illustrated in FIG. 8C, the analyte detection system 800 includes one or more electrodes such as a working electrode 821 and a counter electrode 823. The working electrode 821 be coated, such as gold-coated. The counter electrode 823 may contain carbon or one or more other materials.

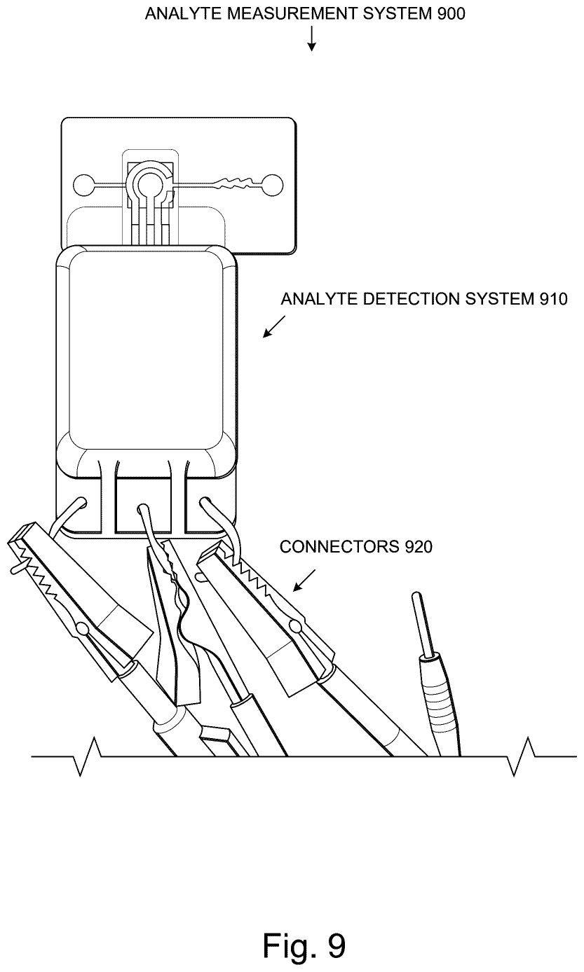

FIG. 9 illustrates an analyte measurement system 900 configured to measure one or more analyte concentrations such as melatonin concentrations. In some aspects, the analyte measurement system 900 includes an analyte detection system 910 and electrical connectors 920 connected to one or more terminals or electrodes of the analyte detection system 910. In some aspects the analyte detection system 910 may be implemented according to any previously described analyte detection systems such as analyte detection systems 110, 210, 410, 500, 600, 700, or 800.

FIG. 10 illustrates a method of measuring analyte concentrations such as melatonin concentrations. In some aspects, an analyte detection system 1000 may be implemented as any previously described analyte detection systems such as analyte detection systems 110, 210, 410, 500, 600, 700, 800, or 900. The analyte detection system 1000 may obtain electrical signals indicative of a number or frequency of interaction events (e.g., between melatonin and a sensing layer). The analyte detection system 100 may be connected to or configured to communicate with one or more hardware, cloud, or edge processors 1010. The analyte detection system 1000 may transmit signals or data indicative of the signals to the processors 1010. The processors 1010 may be configured to generate or output one or more calibration curves, such as the calibration curve 750 or a similar calibration curve. The processors 1010 may normalize electrical signal data 1020 against the calibration curve 750. A parameter of the electrical signal data 1020 may include Tₒₙ, which is a period of time during which an analyte (e.g., melatonin) interacts with a wall of a beta-cyclodextrin pore. Tₒₙ may correspond to a time period starting from a spike or other increase in a current, and finishing when the current returns to a current value immediately before the spike in the current, or returns to a normalized electrical current value that is normalized based on a calibration curve or control curve. Based on the normalized electrical signal data, the processors 1010 may generate a mapping between one or more electrical attributes detected with one or more components such as a working electrode, and an analyte concentration such as a melatonin concentration. The processors 1010 may have electronics and signal processing capabilities having a wide range of sampling rates, signal to noise ratio thresholding, filtering, statistical event detection, or impedance analysis. The processors 1010 may be configured to detect a number of stochastic events or melatonin attachment kinetics.

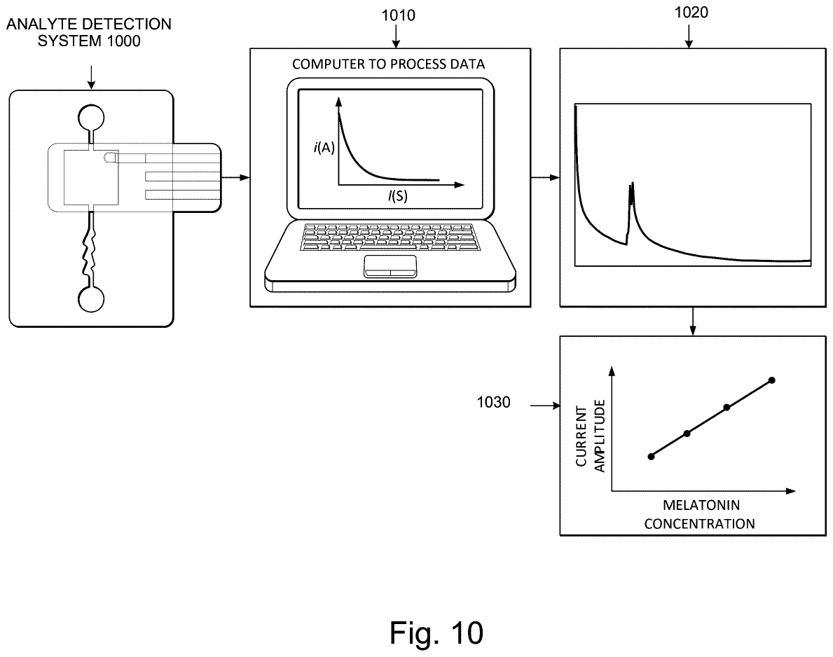

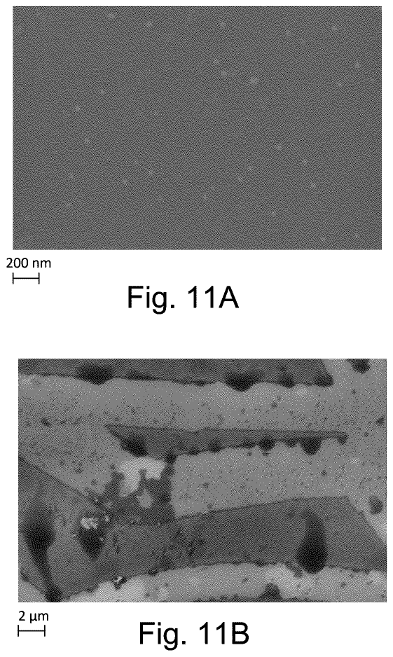

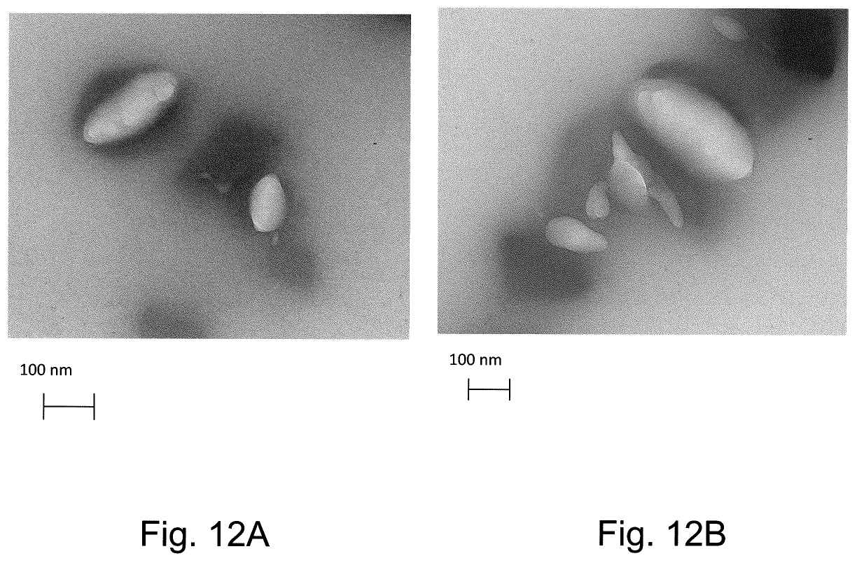

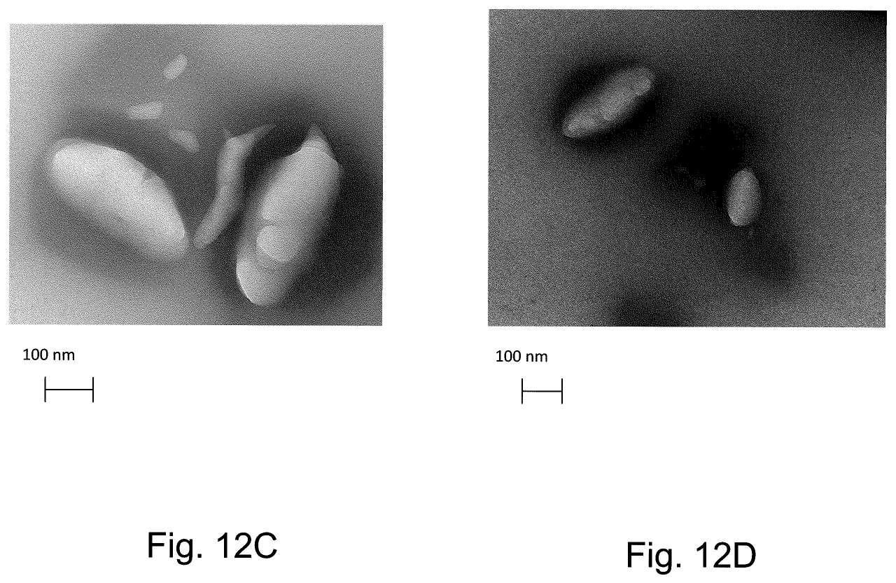

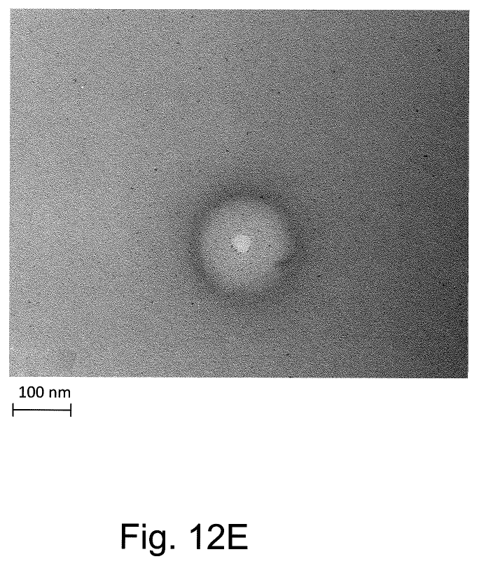

FIG. 11A illustrates an example transduction layer surface, such as a gold sputtered surface. FIG. 11B illustrates an example beta-cyclodextrin functionalized gold surface, in which functionalization may include a Self-Assembled Monolayer (SAM) surface. FIGS. 12A-12E illustrate scanning electron microscopy (SEM) images of beta-cyclodextrin.

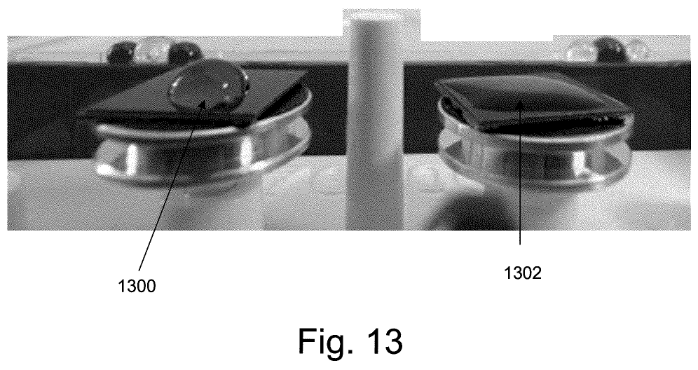

FIG. 13 illustrates examples of assemblies containing a sensing layer (e.g., beta-cyclodextrin) combined with one or more transduction layers. In FIG. 13, an example of an assembly 1300 containing beta-cyclodextrin (e.g., non-functionalized) atop a gold sputtered layer is depicted. An assembly 1302 containing functionalized (e.g., thiolized) SAM surface of beta-cyclodextrin atop a gold sputtered layer is depicted. As shown, the functionalized SAM surface exhibits hydrophilic behavior on the gold sputtered layer which increases sensitivity to fluids and melatonin. Therefore, thiolization of beta-cyclodextrin constitutes a technical benefit of improving detection of melatonin.

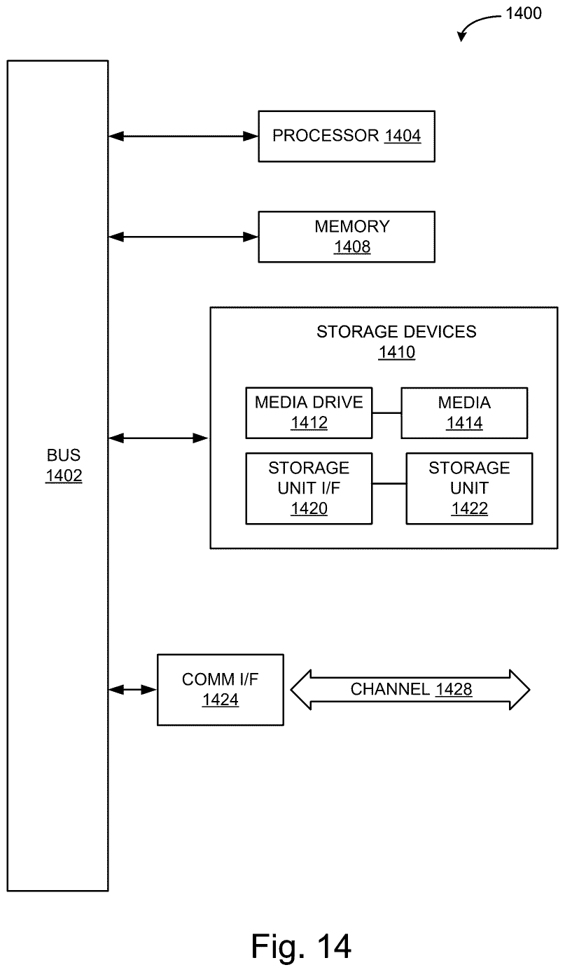

As used herein, the term processor(s) might describe a given unit of functionality that can be performed in accordance with one or more aspects of the present application. As used herein, a processor might be implemented utilizing any form of hardware, software, or a combination thereof. For example, one or more controllers, ASICs, PLAs, PALs, CPLDs, FPGAS, logical components, software routines or other mechanisms might be implemented to make up a processor. Various processors described herein may be implemented as discrete components or described functions and features can be shared in part or in total among one or more processors. In other words, as would be apparent to one of ordinary skill in the art after reading this description, the various features and functionality described herein may be implemented in any given application. They can be implemented in one or more separate or shared processors in various combinations and permutations. Although various features or functional elements may be individually described or claimed as separate components, it should be understood that these features/functionality can be shared among one or more common software and hardware elements. Such a description shall not require or imply that separate hardware or software components are used to implement such features or functionality.

Where processors are implemented in whole or in part using software, these software elements can be implemented to operate with a computing or processing component capable of carrying out the functionality described with respect thereto. One such example computing component is shown in FIG. 14. Various aspects are described in terms of this example-computing component 1400. After reading this description, it will become apparent to a person skilled in the relevant art how to implement the application using other computing components or architectures.

Referring now to FIG. 14, computing component 1400 may represent, for example, computing or processing capabilities found within a self-regulating display, desktop, laptop, notebook, and tablet computers. They may be found in hand-held computing devices (tablets, PDA's, smart phones, cell phones, palmtops, etc.). They may be found in workstations or other devices with displays, servers, or any other type of special-purpose or general-purpose computing devices as may be desirable or appropriate for a given application or environment. Computing component 1400 might also represent computing capabilities embedded within or otherwise available to a given device. For example, a computing component might be found in other electronic devices such as, for example, portable computing devices, and other electronic devices that might include some form of processing capability.

Computing component 1400 might include, for example, one or more processors, controllers, control components, or other processing devices. This can include a processor, or any one or more of the components. Processor 1404 might be implemented using a general-purpose or special-purpose processing engine such as, for example, a microprocessor, controller, or other control logic. Processor 1404 may be connected to a bus 1402. However, any communication medium can be used to facilitate interaction with other components of computing component 1400 or to communicate externally.

Computing component 1400 might also include one or more memory components, simply referred to herein as main memory 1408. For example, random access memory (RAM) or other dynamic memory, might be used for storing information and instructions to be executed by processor 1404. Main memory 1408 might also be used for storing temporary variables or other intermediate information during execution of instructions to be executed by processor 1404. Computing component 1400 might likewise include a read only memory (“ROM”) or other static storage device coupled to bus 1402 for storing static information and instructions for processor 1404.

The computing component 1400 might also include one or more various forms of information storage mechanism 1410, which might include, for example, a media drive 1412 and a storage unit interface 1420. The media drive 1412 might include a drive or other mechanism to support fixed or removable storage media 1414. For example, a hard disk drive, a solid-state drive, a magnetic tape drive, an optical drive, a compact disc (CD) or digital video disc (DVD) drive (R or RW), or other removable or fixed media drive might be provided. Storage media 1414 might include, for example, a hard disk, an integrated circuit assembly, magnetic tape, cartridge, optical disk, a CD or DVD. Storage media 1414 may be any other fixed or removable medium that is read by, written to or accessed by media drive 1412. As these examples illustrate, the storage media 1414 can include a computer usable storage medium having stored therein computer software or data.

In alternative aspects, information storage mechanism 1410 might include other similar instrumentalities for allowing computer programs or other instructions or data to be loaded into computing component 1400. Such instrumentalities might include, for example, a fixed or removable storage unit 1422 and an interface 1420. Examples of such storage units 1422 and interfaces 1420 can include a program cartridge and cartridge interface, a removable memory (for example, a flash memory or other removable memory component) and memory slot. Other examples may include a PCMCIA slot and card, and other fixed or removable storage units 1422 and interfaces 1420 that allow software and data to be transferred from storage unit 1422 to computing component 1400.

Computing component 1400 might also include a communications interface 1424. Communications interface 1424 might be used to allow software and data to be transferred between computing component 1400 and external devices. Examples of communications interface 1424 might include a modem or soft modem, a network interface (such as Ethernet, network interface card, IEEE 802.XX or other interface). Other examples include a communications port (such as for example, a USB port, IR port, RS232 port Bluetooth® interface, or other port), or other communications interface. Software/data transferred via communications interface 1424 may be carried on signals, which can be electronic, electromagnetic (which includes optical) or other signals capable of being exchanged by a given communications interface 1424. These signals might be provided to communications interface 1424 via a channel 1428. Channel 1428 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media. Such media may be, e.g., memory 1408, storage unit 1420, media 1414, and channel 1428. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 1400 to perform features or functions of the present application as discussed herein.

It should be understood that the various features, aspects and functionality described in one or more of the individual aspects are not limited in their applicability to the particular aspect with which they are described. Instead, they can be applied, alone or in various combinations, to one or more other aspects, whether or not such aspects are described and whether or not such features are presented as being a part of a described aspect. Thus, the breadth and scope of the present application should not be limited by any of the above-described exemplary aspects.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

As used herein, a “likelihood” of a traffic condition refers to a computed or estimated probability that a traffic condition will occur at a given location or within a defined region of the transportation network over a specified time horizon. This probability may be derived from any suitable modeling or statistical technique-such as flow-density relationships, historical traffic mappings, machine-learned predictors, or real-time telemetry-and may take into account connected-vehicle attributes (e.g., speed, acceleration, routing intentions), external attributes (e.g., road geometry, signal timing, weather), and any calibrated thresholds. A “likelihood” value exceeding a pre-defined threshold (for example, 0.5 or 50%) is understood to indicate that the traffic condition is predicted to occur with sufficient confidence to warrant implementation of mitigation strategies, whereas a value below that threshold indicates a lower risk of the traffic condition occurring within the defined time horizon.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Reference to A “and” B may be construed to also encompass the scenario of A “or” B. Reference to A “or” B may be construed in an inclusive sense, to also encompass the scenario of A “and” B. Reference to “any of” may be construed to mean “all of,” “some of,” or “one of.” Any reference to “near,” a “threshold” or “sufficiency” may be construed to encompass any applicable value or degree, such as any applicable value or degree sufficient to satisfy a given outcome. In some examples, a threshold level, similarity or degree thereof may be construed to include any values such as 99 percent, 98 percent, 95 percent, 90 percent, 80 percent, 75 percent, or any other value therebetween, or any ranges therebetween. Additionally or alternatively, a threshold similarity, degree, or level may be construed as qualitatively satisfying some condition. For example, a threshold level of detection accuracy may be construed as an absence of a missed detection of an interaction event, or an absence of a false positive or false negative. Reference to “likely,” “a likelihood,” or “probable” or any variation thereof may be construed as satisfying some threshold likelihood or probability.

Additionally, the various aspects set forth herein are described in terms of exemplary block diagrams, flow charts and other illustrations. As will become apparent to one of ordinary skill in the art after reading this document, the illustrated aspects and their various alternatives can be implemented without confinement to the illustrated examples. For example, block diagrams and their accompanying description should not be construed as mandating a particular architecture or configuration.

What is claimed is:

1. An analyte detection system comprising:
an amperometric electrode system comprising a working electrode, the working electrode comprising:
a sensing layer and a transduction layer, the sensing layer being chemically linked to the transduction layer to preserve an orientation of the sensing layer, the sensing layer configured to detect a concentration of an analyte within a fluid medium based on one or more interaction events between the analyte and the sensing layer when the analyte is oriented consistent with the orientation of the sensing layer;
an inlet reservoir from which the fluid medium travels towards the sensing layer; and
an inlet channel through which the fluid medium travels from the inlet reservoir towards the sensing layer, wherein the analyte is oriented consistent with the orientation of the sensing layer when a larger diameter end of the sensing layer is oriented towards the inlet channel or the inlet reservoir.

2. The analyte detection system of claim 1, wherein the sensing layer is unable to detect the analyte when the analyte is oriented inconsistently with the orientation of the sensing layer.

3. The analyte detection system of claim 2, further comprising an outlet channel through which the fluid medium travels from the sensing layer and an outlet reservoir to which the fluid medium travels from the sensing layer, wherein the analyte is oriented inconsistently with the orientation of the sensing layer when a larger diameter end of the sensing layer is oriented towards the outlet channel or the outlet reservoir.

4. The analyte detection system of claim 3, wherein the analyte is oriented inconsistently with the orientation of the sensing layer when a smaller diameter end of the sensing layer is oriented towards the inlet channel or the inlet reservoir.

5. The analyte detection system of claim 1, wherein the analyte comprises melatonin and the sensing layer comprises a thiolized or functionalized calixarenes.

6. The analyte detection system of claim 1, wherein the sensing layer is anchored to an anchoring layer, the anchoring layer comprising a polyacrylic acid brush or a polyacrylic acid film.

7. The analyte detection system of claim 1, wherein the transduction layer comprises a conducting layer and a surface plasmon enhancing layer.

8. The analyte detection system of claim 7, wherein the conducting layer comprises graphite, single or multi walled carbon nanotubes, graphene nanosheets, graphene nanoplatelets, reduced graphene oxide (RGO), buckminsterfullerenes, boron-nitride nanotubes, Mxenes, one-dimensional (1D) nanowires, nanotubes or two-dimensional (2D) nanosheets, metal nanoparticles or metal oxide nanoparticles, a film of noble metals, or metal oxide nanotubes.

9. The analyte detection system of claim 7, wherein the surface plasmon enhancing layer comprises a nanostructured noble metal.

10. The analyte detection system of claim 1, wherein the fluid medium comprises a saliva, a plasma, or urine.

11. A method comprising:
directing a fluid medium containing an analyte from an inlet reservoir through an inlet channel towards a sensing layer of an amperometric electrode system;
detecting, by an amperometric electrode system, a concentration of the analyte within a fluid medium based on one or more interaction events between the analyte and a sensing layer of the amperometric electrode system when the analyte is oriented consistent with an orientation of the sensing layer, wherein the analyte is oriented consistent with the orientation of the sensing layer when a larger diameter end of the sensing layer is oriented towards the inlet channel or the inlet reservoir, and wherein the sensing layer is chemically linked to a transduction layer of the amperometric electrode system to preserve the orientation of the sensing layer.

12. The method of claim 11, wherein the sensing layer is unable to detect the analyte when the analyte is oriented inconsistently with the orientation of the sensing layer.

13. The method of claim 12, further comprising directing the fluid medium from the sensing layer towards an outlet reservoir through an outlet channel, wherein the analyte is oriented inconsistently with the orientation of the sensing layer when a larger diameter end of the sensing layer is oriented towards the outlet channel or the outlet reservoir.

14. The method of claim 13, wherein the analyte is oriented inconsistently with the orientation of the sensing layer when a smaller diameter end of the sensing layer is oriented towards the inlet channel or the inlet reservoir.

15. The method of claim 11, wherein the analyte comprises melatonin and the sensing layer comprises a thiolized or functionalized calixarenes.

16. The method of claim 11, further comprising anchoring the sensor layer to an anchoring layer to preserve the orientation of the sensor layer, wherein the anchoring layer comprises a polyacrylic acid brush or a polyacrylic acid film.

17. The method of claim 11, wherein the transduction layer comprises a conducting layer and a surface plasmon enhancing layer.

18. The method of claim 17, wherein the conducting layer comprises graphite, single or multi walled carbon nanotubes, graphene nanosheets, graphene nanoplatelets, reduced graphene oxide (RGO), buckminsterfullerenes, boron-nitride nanotubes, Mxenes, one-dimensional (1D) nanowires, nanotubes or two-dimensional (2D) nanosheets, metal nanoparticles or metal oxide nanoparticles, a film of noble metals, or metal oxide nanotubes.

19. The method of claim 17, wherein the surface plasmon enhancing layer comprises a nanostructured noble metal.

20. The method of claim 11, wherein the fluid medium comprises a saliva, a plasma, or urine.
